(19) United States
(12) Patent Application Publication
(10) Pub. No.: US 2003/0040478 A1
(43) Pub. Date: Feb. 27, 2003

(54) Chemotherapy treatment

(76) Inventors: Daniel J Drucker, Toronto (CA); Robin P Boushey, Mississauga (CA)

Correspondence Address: FOLEY AND LARDNER, SUITE 500, 3000 K STREET NW, WASHINGTON, DC, 20007, US

(21) Appl. No.: 10/148,682

(22) PCT Filed: Dec. 8, 2000
(86) PCT No.: PCT/IB00/02003

(51) Int. Cl. A61K 38/22; A61K 31/00

(52) U.S. Cl. 514/12; 514/1

(57) ABSTRACT

This invention provides a treatment regimen that is effective in inhibiting chemotherapy-induced apoptosis and promoting cell survival. The invention also relates to a treatment regimen that confers resistance to caspase activation, thereby inhibiting caspase-mediated, proteolytic cleavage of functional cellular enzymes. Specifically, subjects undergoing chemotherapy are first exposed to a pretreatment regimen. Under this regimen, a GLP-2 receptor activator, such as h[GLY2]-GLP2, is administered each day for a predetermined beneficial period, e.g., three consecutive days. Approximately about 1 week following pretreatment, the subjects are exposed to an appropriate chemotherapy treatment regimen. Pretreatment with a GLP-2 receptor activator followed by administration of chemotherapeutic agents improves cell survival, reduces bacteremia, attenuates epithelial injury, and inhibits cellular apoptosis. Moreover, it does not impair the effectiveness of chemotherapy nor result in weight loss. The anti-apoptotic effects of GLP-2 may be useful in the reduction of cytoxicity and bacterial infection induced by chemotherapeutic agents.

### FIELD OF THE INVENTION

The invention relates to methods useful to overcome the damage and adverse effects of chemotherapeutic agents. More particularly, the invention relates to the use of a GLP-2 receptor activator to inhibit chemotherapy-induced apoptosis and promote cell survival in subjects undergoing chemotherapeutic treatment.

### BACKGROUND TO THE INVENTION

Chemotherapeutic agents exert their cytoablative actions on rapidly proliferating cells via several different mechanisms, ultimately leading to cell cycle arrest and/or cellular apoptosis. The cytotoxic actions of chemotherapeutic agents are not tumour-specific and injury to rapidly dividing cells in the bone marrow and intestinal crypt often complicates the treatment of patients with neoplastic disease.

Gastrointestinal toxicity following the administration of chemotherapeutic agents is characterized by severe mucositis, weight loss and systemic infection. Limitation in dose and treatment of chemotherapeutic agents due to gastrointestinal toxicity impair the effectiveness of chemotherapy in susceptible patients. Wadler, S. et al., J. Clin Oncol. 16: 3169-78, 1998. Van Huyen, J. P. et al., Dig. Dis. Sci. 43: 2649-51, 1998. Patients undergoing chemotherapy exhibit a low white blood cell count and can be managed clinically with antibiotics to prevent bacterial infection. Molecules such as granulocyte macrophage-colony stimulating factor (GM-CSF) can also be used to promote restoration of white blood cell count and to attenuate bone marrow toxicity following chemotherapy. Dombret, H. et al., N. Engl. J. Med. 332: 1678-83, 1995.

Several intestine-derived molecules have been identified that maintain the integrity of the mucosal epithelium in part via prevention of apoptosis following intestinal injury. For example, intestinal trefoil factor (ITF) promotes resistance to apoptosis following cellular injury in vitro (Taupin, D. R. et al., Proc. Natl. Acad Sci. USA 97: 799-804, 2000) and ITF-deficient mice exhibit enhanced susceptibility to intestinal injury and increased colonic epithelial cell apoptosis in vivo. Mashimo, H. et al., Science. 274: 262-265, 1996. Similarly, keratinocyte growth factor (KGF) protects mice from chemotherapy and radiation-induced intestinal injury. Farrell, C. L. et al., Cancer Res. 58: 933-9, 1998. In addition, fibroblast growth factor (FGF-2), TGF-β, and several cytokines such as interleukin-11, and interleukin-15 reduce intestinal apoptosis in vivo. Houchen, C. W. et al., Am. J. Physiol. 276: G249-58, 1999; Cao, S. et al., Cancer Res. 58: 3270-3274 1998; Orazi, A. et al., Lab Invest. 75: 33-42, 1996; McCormack, E. S. et al., Biochem. Pharmacol. 53: 1149-59, 1997,

Glucagon-like peptide-2 (GLP-2), a 33 amino acid product of the glucagon gene, is an intestinotrophic peptide secreted by enteroendocrine cells in response to intestinal injury. Taylor, R. G. et al., Baillieres Clin. Endocrinol. Metabolism 8:165-183, 1994; Drucker, D. J. Diabetes 47: 159-169, 1998. Exogenous administration of GLP-2 is trophic to the small and large intestinal epithelium in part via stimulation of crypt cell proliferation. Tsai, C.-H. et al, Am. J. Physiol. 273: E77-E84, 1997. Administration of GLP-2 to rodents with indomethacin-induced intestinal injury improves survival, and reduces epithelial damage, in part via inhibition of apoptosis in the crypt compartment. Boushey, R. P. et al., Am. J. Physiol. 277: E937-E947, 1999. Its use to ameliorate damage to gastrointestinal mucosa caused by CT is suggested in WO96/32414, published Oct. 17, 1996. Similar use of GLP-2 in combination with other growth factors such as growth hormone (GH) and insulin-like growth factors (IGF-1 and IGF-2) is also described in WO99/25644, published Jun. 18, 1998. GLP-2 antagonists also have been described in WO99/03547, published Jan. 29, 1998, for use in pretreating subjects undergoing CT.

Although GLP-2 inhibits apoptosis in the crypt compartment following the administration of indomethacin (Boushey, R. P. et al., Am. J. Physiol. 277: E937-E947, 1999), the mechanisms remain unknown for the coupling of GLP-2 signaling to anti-apoptotic effects.

Cytotoxic stimuli induced by chemotherapeutic agents involve the activation of a class of highly specific proteases called caspases (cysteine-dependent aspartic acid directed proteases). Simizu, S. et al., J. Biol. Chem. 273: 26900-6907, 1998. Studies demonstrating the involvement and importance of the CPP32 (caspase 3) subfamily of caspases in the pathogenesis of irinotecan-induced apoptosis have been reported. Suzuki, A. et al., Exp. Cell Res. 233: 41-7, 1997.

Caspases are synthesized as precursor molecules which require processing at specific aspartate residues to produce the active enzyme. In turn, the enzyme effects highly specific and proteolytic cleavage of functionally important cellular enzymes, such as polyADP ribose polymerase (PARP). Kaufman, S. H. et al., Cancer Res. 53:3976-3985, 1993. Caspases can be grouped into three subfamilies based on their substrate specificities. Group I or the ICE (interleukin 1-α-converting enzyme) subfamily of caspases (caspases 1, 4 and 5), prefer the tetrapeptide sequence WEHD and are believed to play a role in inflammation. Group II (caspases 2, 3 and 7) and group III (caspases 6, 8, 9 and 10) display a specificity for DExD and (I/L/V)ExD, respectively, and are mainly involved in apoptosis. Konopleva, M. et al., Adv. Exp. Med. Biol. 457:217-236, 1999.

### SUMMARY OF THE INVENTION

It is an object of the present invention to provide a treatment regimen that is effective in inhibiting chemotherapy-induced apoptosis and promoting cell survival.

It is also an object of the present invention to provide a treatment regimen characterized by superior benefit to a subject undergoing chemotherapy, by overcoming the damage and adverse effects arising from chemotherapy administration in vivo.

It is also an object of the invention to provide a treatment regimen that confers resistance to caspase activation, thereby inhibiting caspase-mediated, proteolytic cleavage of functionally important cellular enzymes.

Damage and related adverse effects due to chemical insult to the gastrointestinal tract, in particular, by a chemotherapeutic agent, can be reduced significantly by a treatment regimen within the present invention. This regimen entails the pretreatment of the subject with a GLP-2 receptor activator prior to chemical insult. More particularly, and in accordance with an aspect of the invention, a method is provided for treating a subject having a disease or disorder for which treatment by a chemotherapeutic agent is indicated, the method comprising the steps of:

(i) subjecting the subject to a pretreatment regimen effective to alleviate at least one adverse effect of said chemotherapeutic agent, by administering to the subject a GLP-2 receptor activator each day for a prescribed period and

(ii) within about one week following the pretreatment step (i), exposing the pretreated subject to a chemotherapeutic agent treatment regimen appropriate for treating the disease or disorder.

In embodiments, the subject is pretreated with the GLP-2 receptor activator for a period sufficient to protect the subject from the apoptotic effects induced by the subsequently administered chemotherapeutic agent.

In a preferred embodiment, the subject is pretreated with the GLP-2 receptor activator for a period of from two to four consecutive days. In a specific embodiment, the subject is pretreated with the GLP-2 receptor activator for a period of about 3 days.

In a preferred embodiment, the administration of the GLP-2 receptor activator for each day is achieved by twice daily (b.id.) administration of the GLP-2 receptor activator.

In another preferred embodiment, the step of pretreating the subject is performed using the GLP-2 receptor activator as the sole pretreatment agent for alleviating the adverse effects of chemotherapy. Addition of other protective agents, including growth factors such as KGF or KGF-2, is not essential. In alternative embodiments, such addition may be incorporated in the pretreatment regimen if desired.

The superior results achieved with this combination treatment regimen are revealed by the examples presented herein. Most significantly, pretreatment with the GLP-2 receptor activator each day (twice daily dosing) for three consecutive days greatly enhances survival of subjects subsequently receiving chemotherapy. Subject benefits accruing from the method of the present invention are also realized in other terms, including reduced incidence of mucositis, infection, and overall mass and health of gastrointestinal tissue.

Embodiments of the invention, and details thereof, are described hereinbelow with reference to the accompanying drawings.

### BRIEF DESCRIPTION OF THE DRAWINGS

FIG. 1. Animal survival in (A) female CD1 mice treated with two doses of irinotecan (IRT), (B) female BDF1 mice treated with a single dose of 5-fluorouracil (FU), and (C) female tumor-bearing BALB/c mice treated with irinotecan (n=25 mice per treatment group). For experiments shown in A and B, mice were pretreated for 3 days with either saline or h[Gly2]-GLP-2 followed by administration of either two intraperitoneal doses of irinotecan (A) or a single intraperitoneal bolus of 5-fluorouracil (3), **=P<0.01. In protocol (C), repeated 7 day treatment regimens consisting of 3 days of either 0.5 ml saline APBS) or 10 μg h[Gly2]-GLP-2 administered twice daily at 08:00 and 18:00 followed by 3 days of irinotecan (100 mg/kg dose) or vehicle administered once daily followed by a 24 hr recovery period. *=p<0.01, saline versus h[Gly2]-GLP-2/irinotecan-treated mice; #=p<0.01, h[Gly2]-GLP-2 versus h[Gly2]-GLP-2/irinotecan-treated mice; +=p<0.01, saline/irinotecan versus h[Gly2]-GLP-2/irinotecan-treated mice. (D) The tumour inhibition ratio (IR) was determined as described. Kunimoto, T. et al., Cancer Res. 47: 5944-7, 1987. *#=p<0.01 IRT-treated versus non-IRT-treated groups.

FIG. 2. (A) Prevalence of positive bacterial aerobic cultures from mesenteric, splenic, and liver homogenates and whole blood. Groups of mice (n=20 mice for each treatment group) were pretreated for 3 days with either saline or h[Gly2]-GLP-2 as shown in FIG. 1A and euthanized 96 hrs after commencing irinotecan treatment (two injections of 280 mg/kg per dose). *=P<0.05, saline versus h[Gly2]-GLP-2 treated mice after irinotecan. No bacterial colonies were detected in homogenates from control mice (n=5 mice for each treatment group) treated with either saline or h[Gly2]-GLP-2 in the absence of irinotecan. (B) The leukocyte count in saline- and h[Gly2]-GLP-2 treated control and irinotecan-treated mice. + and *=p<0.05 for saline- and h[Gly2]-GLP-2-treated groups versus irinotecan-treated mice. #=P<0.05, irinotecan-treated mice pretreated with saline versus h[Gly2]-GLP-2.

FIG. 3. Mean crypt survival (A and C) and mean cell number per hemi-crypt (B and D) from the midjejunum (A and B) and colon (C and D) of control and irinotecan-treated CD1 mice administered either saline (vehicle) or h[Gly2]-GLP-2 as a 3 day pretreatment regimen. Dotted lines represent line of best fit for data shown between 60 and 96 h. Five mice per treatment group were euthanized for analysis commencing immediately prior to the first of two injections of irinotecan (280 mg/kg per dose) and at 12 hr intervals up to 96 hrs. Crypt survival was measured along the entire bowel circumference, and the mean cell number per hemi-crypt determined in 50 consecutive intact crypts per animal. *=P<0.05, **=P<0.001, saline versus h[Gly2]-GLP-2 treated mice.

FIG. 4. Positional detection of apoptotic cells in the crypt compartment using TdT-mediated dUTP nick-end labeling (TUNEL assay). Apoptotic scores were determined from mid-jejunal (A) and colonic (B) intestinal crypt compartments of irinotecan-treated mice by determining the total number of TUNEL-positive cells in 50 continuous crypts (n=5 animals/treatment group). *=P<0.05 and **=P<0.01, saline versus h[Gly2]-GLP-2 treatment. Analysis was performed by calculating the % of TUNEL-positive cells in each crypt-cell position for all intact crypts present in a single transverse intestinal cross-section, as previously described. Pritchard, D. M. et al., Cancer Res. 58: 5453-65, 1998; Potten, C. S. et al. Br. J. Cancer 78: 993-1003, 1998; Ijiri, K. et al., Br. J. Cancer 47: 175-85, 1983. Five mice were analyzed at each time point per treatment group. *=P<0.05, **=P<0.01, saline versus h[Gly2]-GLP-2 treated mice. Dark shaded box represents the stem cell region (SCP) whereas the light shaded box represents the clonogenic potential stem cell region (CPSC). Pritchard, D. M. et al., Cancer Res. 58: 5453-65, 1998; Potten, C. S. et al., Br. J. Cancer 78: 993-1003, 1998; Ijiri, K. et al., Br. J. Cancer 47: 175-85, 1983. (C). Analysis of procaspase 8 (proC 8) cleavage to the active p18 subunit (C 8) by Western blotting in the colon of mice following irinotecan (IRT) treatment. *=p<0.05, **=p<0.01, vehicle versus h[Gly2]-GLP-2.

FIG. 5. Irinotecan induced apoptosis in a Baby Hamster Kidney (BHK) fibroblast cell line containing the stably integrated pcDNA3.1 plasmid (BHK-pcDNA3) or the identical plasmid directing expression of the rat GLP-2 receptor (BHK-GLP-2R). (A) Analysis of cell viability in BHK-pcDNA3 and BHK-GLP-2R cells, respectively, following irinotecan treatment. Values are derived from experiments performed in quadruplicate. *=P<0.05, irinotecan alone versus irinotecan/h[Gly2]-GLP-2. (B) Analysis of caspase-like activity in BHK-GLP-2R cells following treatment with irinotecan (IRT). Ac-IETD-pNA (caspase-8-like activity) and Ac-LEHD-pNA (caspase-9-like activity) are represented as fold-induction compared to cells not treated with irinotecan. *=P<0.05, h[Gly2]-GLP-2/IRT versus IRT. (C) Cleavage of Ac-DEVD-pNA (caspase-3-like enzyme activity) and procaspase-3 in irinotecan-treated cells treated with or without h[Gly2]-GLP-2 or forskolin (FK). *=P<0.01, IRT alone versus IRT/forskolin or IRT/h[Gly2]-GLP-2. (D) Western blot analysis of PARP cleavage in irinotecan-treated BHK-GLP-2R cells. *=p<0.05 for IRT alone versus IRT/forskolin or IRT/h[Gly2]-GLP-2. For data in FIGS. 5B-5D, values are expressed as fold induction relative to untreated cells. The relative densitometric values for caspase-3 (C) or PARP (D) were normalized to the values obtained for actin in the same experiments and represent the means of 3-4 separate experiments.

FIG. 6. Schematic representation of how GLP-2R-dependent signaling regulates irinotecan-induced apoptosis in the intestine in vivo and in cells expressing the transfected GLP-2 receptor (BHK GLP-2R) in vitro.

### DETAILED DESCRIPTION OF THE INVENTION

The invention relates to a method for treating a subject with a therapeutic regimen effective to inhibit chemotherapy-induced apoptosis and to promote cell survival. The invention also relates to a treatment regimen that confers resistance to caspase activation thereby inhibiting caspase-mediated proteolytic cleavage of functional cellular enzymes.

One aspect of the present invention relates to a method for treating subjects who are about to undergo chemotherapy for the treatment of cancer and other diseases, characterized by uncontrolled cell or tissue proliferation. Another aspect of the invention relates to a method of treatment for subjects receiving cytotoxic agents such as biocides (e.g., anti-virals, anti-fungals and anti-bacterials) causing adverse effects on the gastrointestinal tract.

One aspect of present invention can be applied to ameliorate the adverse effects due to chemical insult. Useful applications covered by the present invention include inhibition of chemically-induced development of intestinal mucositis, reduction of the incidence and severity of infection, inhibition of white blood cell depletion, and damage resistance to the large bowel.

Another aspect of the present invention is to ameliorate the adverse effects of chemical insult in the functioning of the small bowel, e.g., to improve the incidence of malabsorption, ulceration, bleeding, infection, diarrhea, and fibrosis and stricture formation leading to reduced length and function of the small bowel.

The subjects used in the present invention apply to humans and to various animals for veterinary purposes including pets and livestock.

The method of the present invention utilizes a GLP-2 receptor activator and any cytotoxic chemical agent. The term “GLP-2 receptor” refers to a cell surface protein that binds to and is activated by glucagon-like peptide 2 (GLP-2). In structural terms, the GLP-2 receptor is a G-protein coupled receptor having the characteristic pattern of seven transmembrane domains. In terms of the amino acid sequence, the GLP-2 receptor is represented by the human homolog reported by Munroe D. G. et al., Proc. Natl. Acad. Sci. USA 96:1569-1573, 1999. GLP-2 receptor agonists useful in the present method are those that activate the GLP-2 receptor by binding to that receptor and thereby stimulating an intracellular second messenger system coupled to that receptor.

The terms “GLP-2 receptor activator” refers to any agent that activates the GLP-2 receptor, and includes GLP-2 receptor agonists, and GLP-2 receptor modulators. The “GLP-2 receptor modulators” are agents that participate in GLP-2 receptor activation in an allosteric manner, typically by binding at a site on the GLP-2 receptor other than the agonist binding site. The GLP-2 receptor modulators thus influence triggering of the GLP-2 receptor by an agonist, to cause a modulated and desirably up-regulated activation of the receptor. GLP-2 receptor modulators thus can be identified as those agents that alter or modulate the activity of the GLP-2 receptor. A “GLP-2 receptor agonist” is an agent that triggers directly the GLP-2 receptor, to stimulate biochemical cascades coupled intracellularly to the GLP-2 receptor. GLP-2 receptor agonists thus are agents that bind directly to the GLP-2 receptor binding site, and therefore, unlike GLP-2 receptor modulators, can be competitively inhibited by GLP-2 receptor antagonists.

The GLP-2 receptor activators can be identified rapidly by screening compound and peptide libraries against cells engineered genetically to produce the GLP-2 receptor. Cells functionally incorporating the GLP-2 receptor and their use to screen compounds for GLP-2 receptor activators, are also described in WO98/25955, published Jun. 18, 1998. The materials and methods therein described are useful to identify the GLP-2 receptor activators useful in the present invention. Applications of the GLP-2 receptor cell lines in drug screening further are described in WO053208 published Sep. 14, 2000.

Desirably, the GLP-2 receptor activator is an agent that reduces chemotherapy-induced activation of the caspase pathway which leads to apoptosis. Assays suitable for identifying such agents are exemplified herein.

In a preferred embodiment, the GLP-2 receptor activator is a GLP-2 receptor agonist.

In one embodiment of the invention, the GLP-2 receptor agonist is a compound that acts selectively at the GLP-2 receptor. Selectively-acting GLP-2 receptor agonists are compounds that, in the context of a suitable GLP-2 receptor binding or functional assays, bind to the GLP-2 receptor with greater affinity, desirably at least an order of magnitude greater affinity, relative to different receptor types, such as the GLP-1 receptor. In other embodiments, the GLP-2 receptor agonist is a compound that binds to the GLP-2 receptor with an affinity at least equivalent to the affinity of GLP-2.

In one embodiment, the GLP-2 receptor activator is a small molecule GLP-2 receptor agonist, of the type described for instance in WO053208 published Sep. 14, 2000. Specific small molecule GLP-2 receptor agonists include 2-Methyl-5-[(4-(phenyl ethynyl)]phenyl oxazole; 2-Phenyl-5-[(4-(phenyl ethynyl)]phenyl oxazole; 2-(Benzoylamino)-α-[(4-chlorobenzylidene)hydrazino]benzaldehyde; 2-(Benzoylamino)-α-[(4-dimethylaminobenzylidene)hydrazino]benzaldehyde; and 2-(4-Chloro-benzoylamino)-α-[((4-hydroxy-3-methoxy)benzylidene)hydrazino]benzaldehyde.

In another embodiment of the present invention, the GLP-2 receptor agonist is a wild-type GLP-2 peptide. It can include any vertebrate GLP-2 peptide such as chicken and trout. In particular, it can include human GLP-2 and mammalian homologs thereof such as primate, rat, mouse, porcine, oxine, bovine, degu, hamster, and guinea pig GLP-2. Desirably, but not essentially, the GLP-2 selected for use is of the same species as the subject identified for treatment by chemotherapy.

In other embodiments of the invention, the GLP-2 receptor agonist is an analog of wild type GLP-2 that incorporates one or more amino acid substitutions, additions, deletions or modifications. Agonist activity of human GLP-2 and rat GLP-2 is believed to require an intact N-terminus, but various deletions of up to several residues at the C-terminus are tolerated without the loss of agonist activity. Substitutions are tolerated at sites outside regions conserved across the various GLP-2 species homologs. Similarly, substitutions are also tolerated at sites within regions conserved across GLP-2 species. Accordingly, in some embodiments, the amino acid substitutions are conservative substitutions, for instance, in which one member of an amino acid class is substituted by another member, e.g., the substitution of alanine by glycine, the substitution of asparagine by glutamine, the substitution of methionine by leucine or isoleucine and the like.

The GLP-2 receptor activator may further incorporate modifications that improve the biological and other properties of the compound. For instance, serum half-life may suitably be enhanced, such as by derivatization with fatty acid and other moieties at the epsilon amino groups of internal lysines, as described for instance in WO98/08872, published Mar. 5, 1998.

In a preferred embodiment, the GLP-2 receptor is a GLP-2 analog that has been altered to confer resistance to degradation by endogenous enzymes, such as DPP-IV. Such analogs suitably incorporate a replacement of an alanine residue at position 2. In specific embodiments, the Ala2 residue is replaced by glycine or serine, or by other residues as described in U.S. Pat. No. 5,789,379. In a most preferred embodiment, the GLP-2 receptor agonist is [Gly²]GLP-2. For use in treating humans, the GLP-2 receptor agonist is desirably but not essentially a human GLP-2 peptide or analog, particularly including the Gly2 analog of human GLP-2.

The method of the invention utilizes any GLP-2 receptor activator and any cytotoxic chemical agent useful medically to treat a given condition, disease or disorder. Included among such cytotoxic chemical agents are chemotherapeutic agents such as 5-fluorouracil (5-FU), irinotecan (IRT), BCNU, busulfan, carboplatin, daunorubucin, doxorubicin, etoposide, gemcytabine, ifosphamide, melphalan, methotrexate, navelbine, topotecan, taxol, taxotere, and useful combinations of these. In the method of the present invention, these chemotherapeutic agents are employed in the manner already established for their use in treatment of the given condition, such as cancer treatment. These could be in terms of dosage, delivery route, and treatment protocol. In accordance with the present invention, however, subjects receiving such treatment are first pretreated with a GLP-2 receptor activator.

For use in the present method, the GLP-2 receptor activator may be formulated for delivery by injection or otherwise, in accordance with established practice. In embodiments of the invention, the activator is formulated for delivery by single or repeated subcutaneous or intravenous injection, or by intravenous or subcutaneous infusion. Pharmaceutically acceptable aqueous vehicles include phosphate-buffered saline. Incorporated herein by reference: UK Patent Application No. 9930882.7.

For use in pretreating a subject in accordance with the present invention, the GLP-2 receptor activator is administered to the subject on a daily basis for a predetermined period prior to administration of the chemotherapeutic agent. The pretreatment period most suitable for human subjects can be determined by clinical trial. Suitable pretreatment periods are identified as those providing a given benefit to the subject, relative to subjects not pretreated with GLP-2 receptor, in terms of any one of these endpoints following chemotherapy: enhanced survival, improved small or large bowel health or function, higher white blood cell count, reduced incidence of infection or bacterial count, and incidence of mucositis.

It is anticipated that such benefits will results from a pretreatment regimen in which the GLP-2 receptor activiator is administered for a period sufficient to activate, within GLP-2 receptor-presenting cells, a biochemical cascade responsible for inhibiting activation of caspases, and particularly caspase-8 and caspase-3. Thus, subjects desirably are pre-treated with the GLP-2 receptor activator for a period sufficient to protect GLP-2 receptor-presenting cells from the induction of caspase enzymes by the subsequently administered chemotherapeutic agent.

In a preferred embodiment of the invention, the pretreatment period consists of from two days to four days. In a specific embodiment, the pretreatment period consists of three consecutive days of pretreatment with a GLP-2 receptor activator.

In an embodiment of the invention, administration of the daily dose of GLP-2 receptor activator can be accomplished either by administering a single dose constituting the desired total daily dose, or by administration of two or more individual doses to the subject each day to attain the desired daily dose. In a preferred embodiment, the subject is treated twice daily to deliver the total daily dose, for instance by delivering half the total daily dose in the morning, and half the total daily dose in the evening. The time between dosing is not critical, and is suitably within about 8-16 hours, e.g., about 12 hours. Alternatively, the subject can be dosed three or four times a day, or more often, if desired, to introduce the desired total daily dose. An appropriate total daily dose will, of course, vary with the species of the subject to be treated, together with the age, weight, gender, and medical condition of the subject. Suitable daily doses can be determined from the rodent models and results herein presented, and can be refined further in appropriately controlled clinical trials. Endpoints useful in those clinical trials to identify suitable doses will include such differences, relative to subject baseline, as increased proliferation of gastrointestinal tissue as determined by duodenal or rectal biopsy after several days of GLP-2 receptor activator treatment, and increased nutrient absorption following nutrient challenge. It is anticipated that an appropriate daily dose will lie in the range from about 1 μg/kg to about 1 mg/kg. For humans, a suitable total daily dose is anticipated to lie within the range from 10 μg to about 100 mg, and more particularly, in the range from 100 μg to 50 mg, e.g., 1-10 mg.

In accordance with the present method, pretreatment with the GLP-2 receptor activator is followed “within about one week” and preferably within not more than about 5 or 6 days, by commencement of treatment with the chemotherapeutic or other chemical agent. The interval between pretreatment with GLP-2 and commencement of chemotherapy can be determined more accurately during appropriately designed clinical trials, and endpoints typical of chemotherapy trials such as tumor regression, reduced rates of proliferation or metastasis, etc. On the basis of the rodent studies herein exemplified, it is appropriate that chemotherapy treatment commences within about 3 days, e.g., within 48 hours, following pretreatment. The protective and proliferative effects of the GLP-2 receptor activator may regress within about one week following final dosing; accordingly, such chemotherapeutic treatment preferably commences on the day following completion of the pretreatment regimen, e.g., within about 8-36 hours, and desirably within about 12-24 hours, following pretreatment, and is performed in accordance with a regimen established for the given chemotherapeutic. Such treatment may also include radiation therapy, as an adjunct to chemotherapy.

The particular chemotherapeutic regimen will be determined by the type of chemotherapeutic agent selected for use and the type of cancer being treated. For instance, head and neck cancer patients may receive radiation therapy together with 5-fluorouracil and cisplatinum over a seven week period. Colorectal cancer patients may receive 5-fluorouracil with leucovorin each day for five days. In particular embodiments, for subjects with advanced colorectal cancer, a suitable chemotherapy regimen may consist of one of the following regimens based on 5-fluorouracil (5-FU): (1) weekly 5-FU 600 mg/m² i.v. bolus; (2) weekly 5-FU 600 mg/m² bolus plus leucovorin (LV) 500 mg/m² 2-h i.v. infusion; (3) weekly 5-FU 2600 mg/m² 24-h continuous i.v. infusion plus LV 100 mg 4-h i.v. infusion and 50 mg orally every 4 h for five doses. An all-oral regimen of etoposide and cyclophosphamide can be administered to subjects with poor-prognosis extensive disease small-cell lung cancer. This entails administration either of (1) etoposide orally at 50 mg daily and cyclophosphamide orally at 50 mg daily days 1-14 every 28 days, or both agents orally at 50 mg twice daily days 1-14 every 28 days. In subjects with metastatic breast cancer, treatment can entail administration of vinorelbine 20 mg/m² on day 1, doxorubicin 40 mg/m² on day 1, methotrexate 100 mg/m² on day 1 and leucovorin 20 mg orally every 6 h for six doses beginning on day 2. Treatment can be repeated every 21 days.

As is exemplified hereinafter, the pretreatment regimen appears not to require adjustment relative to the type of chemotherapeutic agent selected for subsequent subject treatment. Excellent and consistent results have been obtained when chemotherapeutic treatment using either 5-FU or irinotecan (CPT-11 or Camptothecin) follows pretreatment with a GLP-2 receptor activator. The benefits accruing from the present pretreatment regimen are pronounced particularly when the chemotherapeutic agent is given orally or by another enteral delivery route, resulting in direct insult to the gastrointestinal tract, although benefits such as enhanced white blood cell count are also obtained when the chemotherapeutic agent is delivered parenterally.

In the event that a given subject requires more than one episode of chemotherapeutic treatment, that subject can again be pretreated with GLP-2 receptor and activator in accordance with the present method if more than about one week, e.g. seven or more days, elapses between completion of one chemotherapy episode and start of the next chemotherapy episode.

The following examples are provided to illustrate the specific aspects of the present invention and should not be construed to limit the scope of the present invention.

### EXAMPLE 1

Effect of Pretreatment of GLP-2 Receptor Activator on Animal Survival Prior to Chemotherapeutic Administration

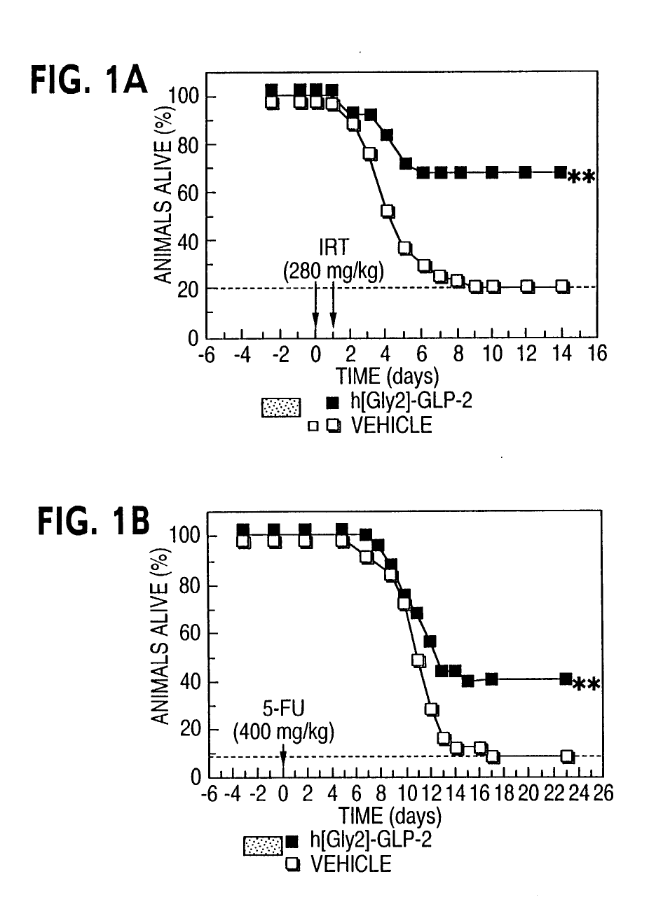

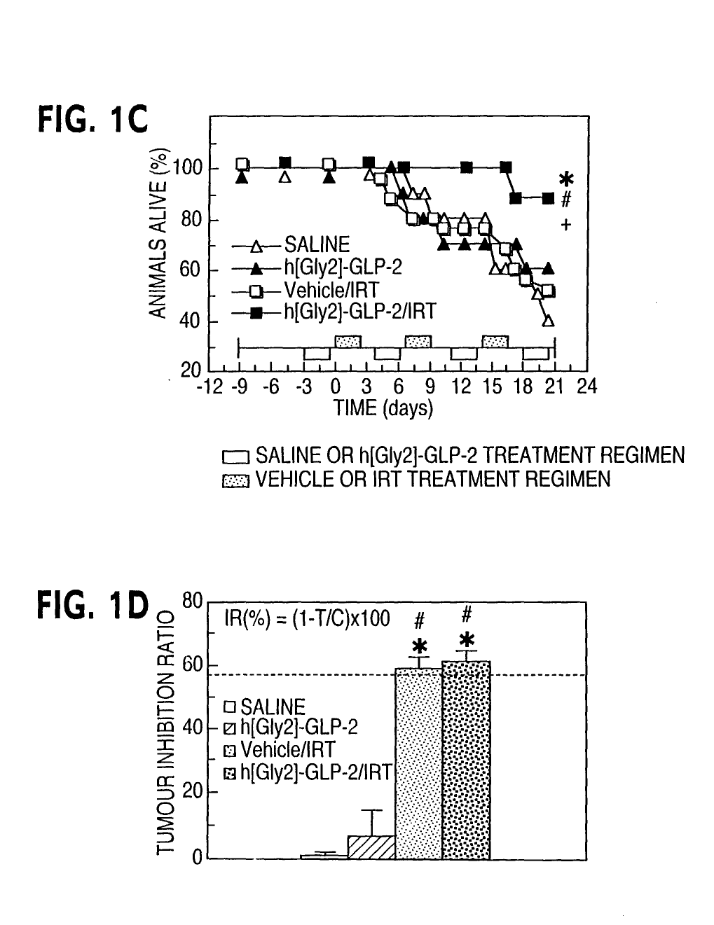

This example demonstrates that administration of h[Gly2]-GLP-2 for 3 days prior chemotherapeutic administration significantly enhanced animal survival. This was observed in CD1 mice treated with irinotecan (FIG. 1A). The protective effect of h[Gly2]-GLP-2 was not restricted to a single chemotherapeutic agent or murine genotype. Similarly, significant enhancement of survival was demonstrated in BDF-1 mice following administration of the anti-metabolite 5-fluorouracil (5-FU) was also observed (FIG. 1B).

To assess the efficacy of h[Gly2]-GLP-2 in tumor-bearing mice, CT-26 murine colon carcinoma tumour cells were inoculated into BALB/c mice and propagated in vivo. h[Gly2]-GLP-2 enhanced survival following cyclical irinotecan administration in tumour-bearing mice (FIG. 1C, p<0.01, h[Gly2]-GLP-2/irinotecan versus all other groups of mice). Mice receiving both h[Gly2]-GLP-2 and irinotecan (100 mg/kg) tolerated three times the amount of irinotecan before equivalent rates of mortality were observed (FIG. 1C). Furthermore, tumor-bearing mice treated with h[Gly2]-GLP-2 and irinotecan exhibited significantly less weight loss compared to mice receiving irinotecan alone (p<0.05; data not shown). Although h[Gly2]-GLP-2 enhanced survival and reduced weight loss, it did not impair irinotecan-induced tumour regression (FIG. 1D). The above findings further shows that the protective effects of GLP-2 were not diminished in the setting of active neoplasia.

### EXAMPLE 2

Effect of GLP-2 Receptor Activator on Bacteremia and Leucocyte Count Following Chemotherapeutic Administration

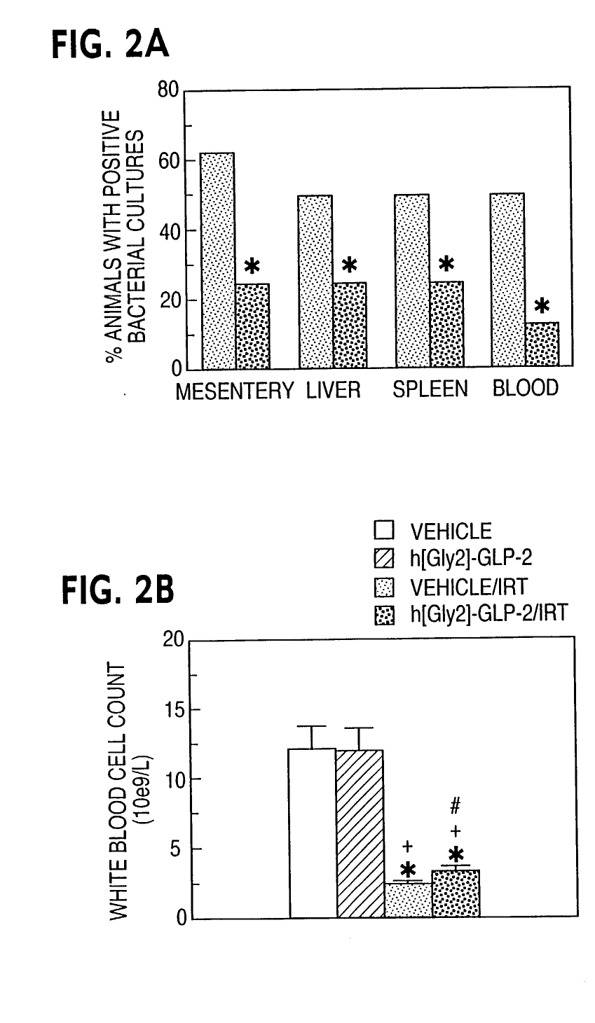

This example shows the efficacy of GLP-2 receptor activator in reducing bacteremia and increasing white blood cell count after chemotherapeutic treatment. As chemotherapy administration may be associated with increased intestinal permeability and bacterial septicemia, bacterial infection in chemotherapy-treated mice was assessed. h[Gly2]-GLP-2-treated mice exhibited a significant reduction in bacterial culture positivity in all organs examined 96 hrs following irinotecan administration (FIG. 2A, P<0.05 for saline versus h[Gly2]-GLP-2 after irinotecan. A significant leukopenia was observed in mice following irinotecan treatment. Mean white blood cell count was modestly but significantly higher in h[Gly2]-GLP-2-treated mice (FIG. 2B).

The significant reduction in chemotherapy-associated mortality in h[Gly2]-GLP-2-treated mice may be explained in part by the reduction in circulating bacteremia. Recent reports have demonstrated that GLP-2 reduces mucosal permeability in rats following major small bowel resection. Scott, R. B. et al., Am. J. Physiol. 275: G911-G921, 1998. Furthermore, GLP-2 markedly reduced circulating bacteremia and decreased bacterial infection in the liver and spleen in mice following indomethac-induced intestinal injury. Boushey, R. P. et al., Am. J. Physiol. 277: E937-E947, 1999. Although the precise mechanism(s) activated by h[Gly2]-GLP-2 leading to reduction in bacterial infection remains unknown, the demonstration that h[Gly2]-GLP-2 reduced macromolecular flux, decreased intestinal permeability, and markedly enhanced intestinal barrier function in GLP-2 mice (Benjamin, M. A. et al., Gut 47: 112-119, 2000) provides a clear link between GLP-2 action and reduced bacterial translocation in the setting of intestinal injury. Therefore, it seems likely that GLP-2-mediated enhancement of intestinal barrier function contributes to the reduction in bacterial sepsis observed following irinotecan in h[Gly2]-GLP-2-treated mice.

### EXAMPLE 3

Effect of GLP-2 Receptor Activator on Crypt Loss After Chemotherapy Treatment

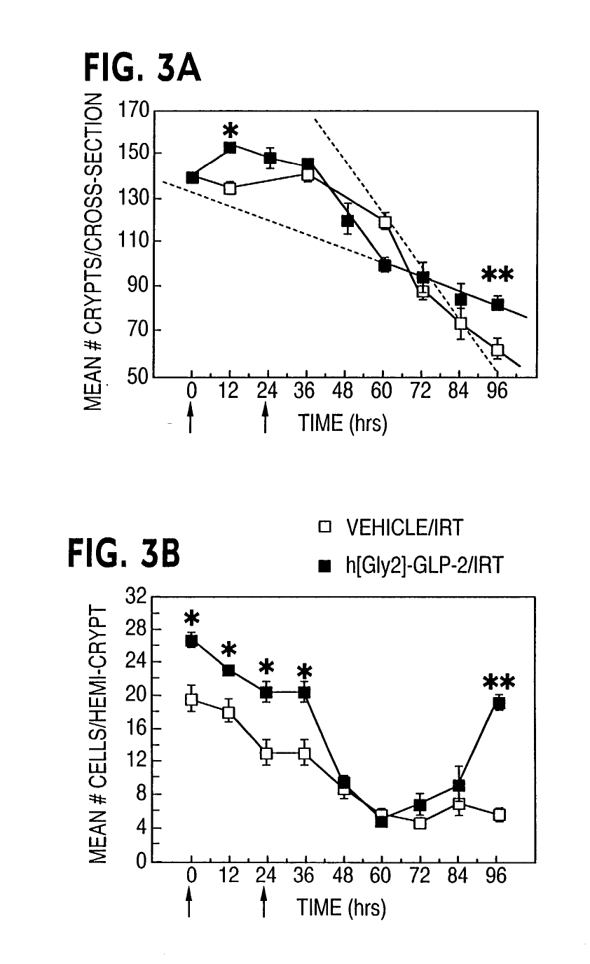

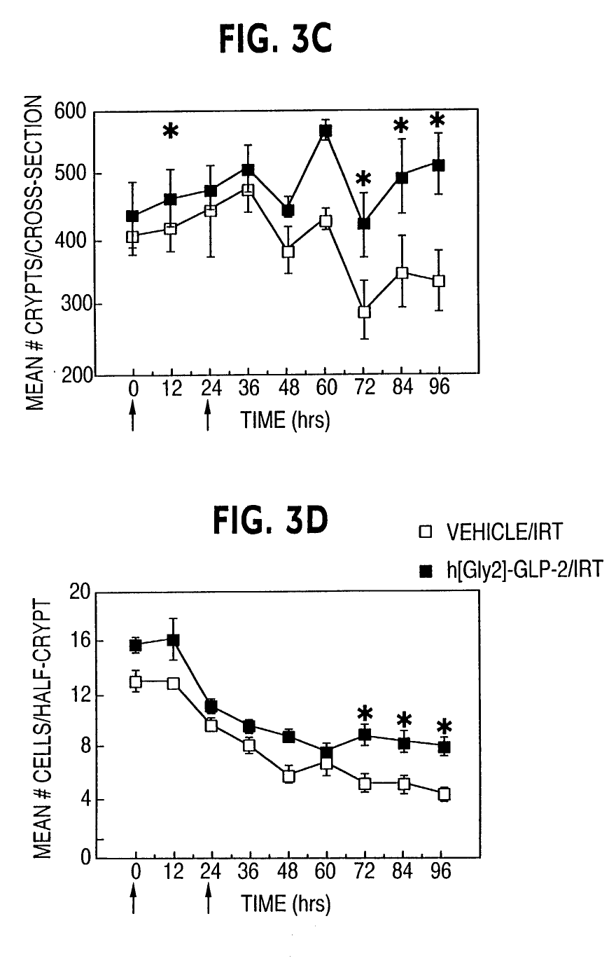

This example describes the histological consequences of h[Gly2]-GLP-2 action in the setting of chemotherapy. The crypt compartment of irinotecan-treated mice were analyzed. Morphometric analysis revealed a significant reduction in both the number of crypts and in the number of cells within each crypt in the small and large intestine following irinotecan (FIGS. 3A-D). h[Gly2]-GLP-2 significantly reduced the rate of crypt loss in the jejunum (FIG. 3A) and restored crypt cell number 96 hrs following irinotecan (FIG. 3B). Similarly, h[Gly2]-GLP-2 pretreatment prevented crypt loss and enhanced crypt cell number in the colon (FIGS. 3C, 3D).

The initial observation that GLP-2 exerts trophic actions in the intestinal mucosa was largely attributed to stimulation of crypt cell proliferation, Drucker, D. J. et al., Proc. Natl. Acad. Sci. USA. 93: 7911-7916, 1996; Tsai, C.-H. et al., Am. J. Physiol. 273: E77-E84, 1997. Although the number of identifiable cells undergoing spontaneous apoptosis in the normal intestinal crypt is low, intestinal injury following exposure to ionizing radiation, or chemical agents resulted in marked induction of apoptosis in the crypt compartment. Farrell, C. L. et al., Cancer Res. 58: 933-9, 1998; Houchen, C. W. et al., Am. J. Physiol. 276: G249-58, 1999; Cao, S. et al., Cancer Res. 58: 3270-3274, 1998; Pritchard, D. M. et al., Cancer Res. 58: 5453-65, 1998; Potten, C. S. et al., Br. J. Cancer 78: 993-1003, 1998; Ijiri, K. et al., Br. J. Cancer 47: 175-85, 1983; Coopersmith, C. M. et al., Oncogene 15: 131-41, 1997; Watson, A. J. et al., Am. J. Physiol. Gastrointest. Liver Physiol. 278: G1-G5, 2000. The above findings that GLP-2 reduces the percentage of apoptotic cells in the crypt compartment following chemotherapy is consistent with recent evidence demonstrating a marked reduction in crypt apoptosis following GLP-2 treatment of mice with indomethacin-induced enteritis. Boushey, R. P., et al., Am. J. Physiol. 277: E937-E947, 1999. Therefore, the above results suggest that GLP-2 can maintain the integrity of the intestinal epithelium by stimulating cell proliferation and inhibiting apoptotic cell death in the crypt compartment.

### EXAMPLE 4

Temporal and Spatial Analysis of Crypt Apoptosis Following Chemotherapeutic Injury

The purpose of this example is to understand the mechanisms by which h[Gly2]-GLP-2 protected the cells underlining the crypt compartment of the small and large intestine from irinotecan-induced injury. A temporal and spatial analysis of apoptosis in the crypt compartment was initially performed. Accordingly, pluripotent stem cells (SC) within the crypt compartment are thought to reside at cell positions 3-5 in the small intestine, and at positions 1-3 in the colon, while the clonogenic potential stem cells (CPSC) reside at positions 6-8 in the small intestine, and at positions 5-7 in the colon. Potten, C. S. et al., Br. J. Cancer 78: 993-1003, 1998; Ijiri, K. et al., Br. J. Cancer 47: 175-85, 1983.

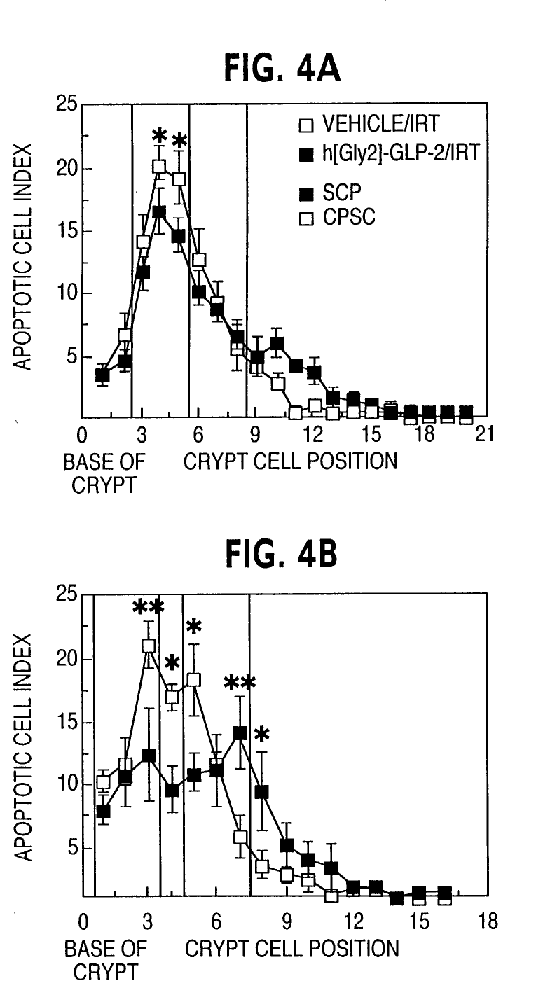

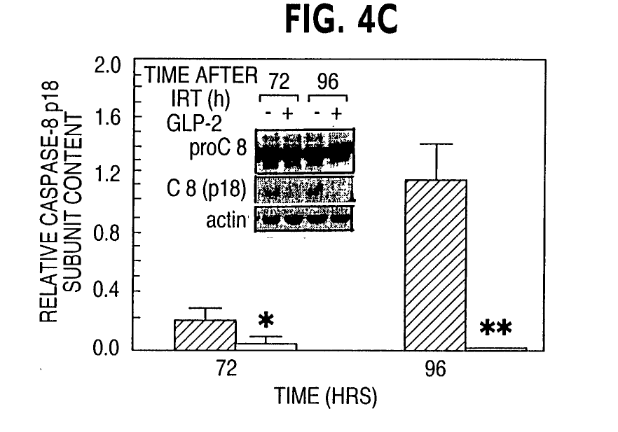

Based on the above information, a positional topographical assessment of apoptosis within the crypt compartment was performed. h[Gly2]-GLP-2 pretreatment significantly reduced apoptosis in the jejunum at crypt cell positions 4-5 (FIG. 4A, P<0.05, saline versus h[Gly2]-GLP-2 treated mice). Similarly, h[Gly2]-GLP-2 reduced apoptosis in the colon at crypt cell positions 3-5 (FIG. 4B, P<0.05, saline versus h[Gly2]-GLP-2 treated mice). These results provide further support that GLP-2 can maintain the integrity of the intestinal epithelium by stimulating cell proliferation and inhibiting apoptotic cell death in the crypt compartment.

Furthermore, a significant reduction in procaspase-8 cleavage was observed in the colon of h[Gly2]-GLP-2-treated mice at both 72 and 96 h after irinotecan (FIG. 4C, *=P<0.05 and **=P<0.01 for saline versus h[Gly2]-GLP-2 treated mice receiving irinotecan).

### EXAMPLE 5

Direct Effects of GLP-2 on Apoptosis in vitro

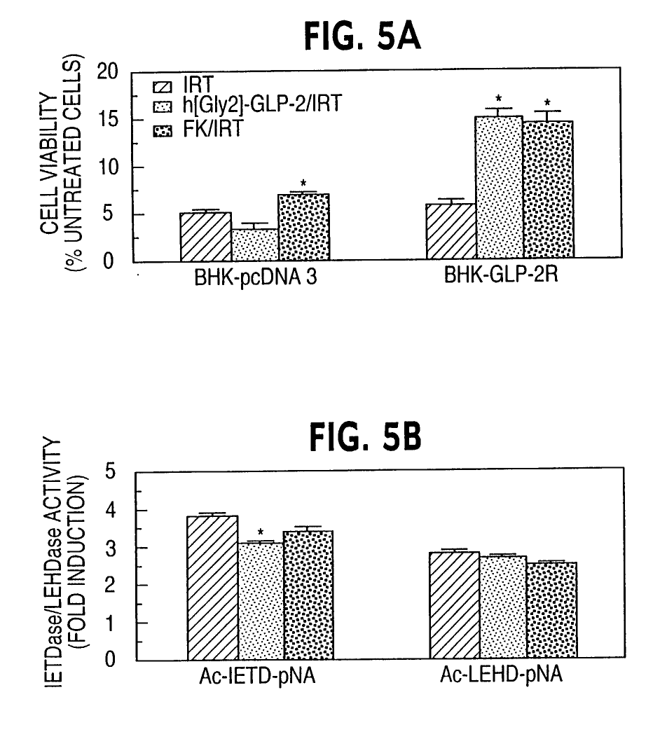

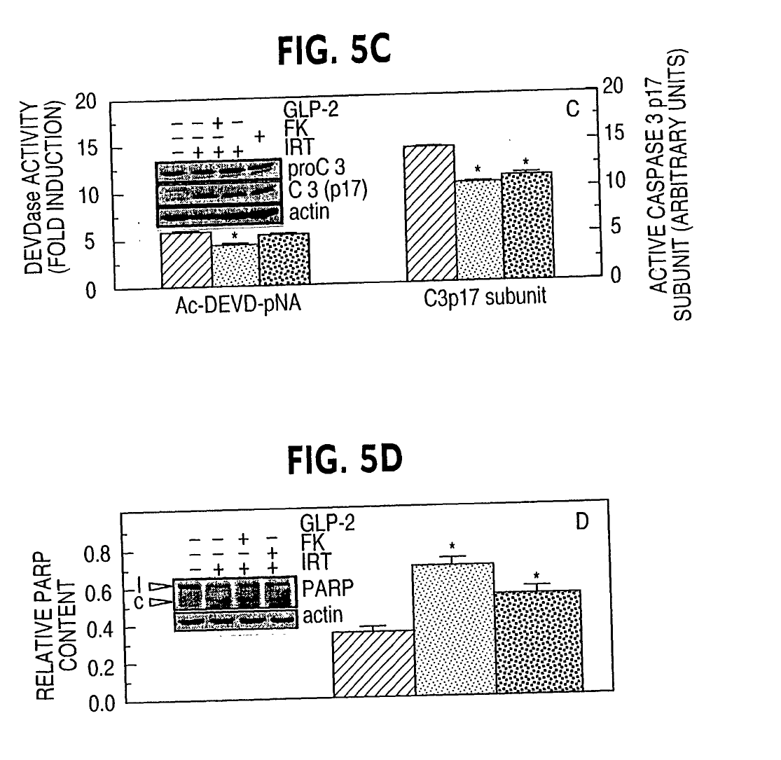

The purpose of this example is to understand the mechanisms activated by GLP-2 receptor signaling in conferring resistance to apoptosis-mediated gastrointestinal injury following irinotecan treatment. The small and large intestine is comprised of a mixed heterogeneous population of cell types that may be differentially affected by irinotecan. Since intestinal cell lines expressing the endogenous GLP-2 receptor have not yet been identified, BHK cells expressing the rat GLP-2 receptor (Yusta, B. et al., J. Biol. Chem. 274: 30459-67, 1999) were used to examine the direct effects of GLP-2 on apoptosis in vitro. A significant improvement in cell viability was observed following pretreatment of BHK-GLP-2R cells, but not control BHK cells, following pretreatment with h[Gly2]-GLP-2 for 36 hrs prior to irinotecan administration (FIG. 5A). Analysis of caspase-8 and caspase-9-like protease activity following irinotecan treatment was quantified by assessing cleavage of the substrates Ac-IETD-pNA and Ac-LEHD-pNA, respectively. h[Gly2]-GLP-2 treatment significantly reduced caspase-8-like enzymatic activity (FIG. 5B; p<0.05). In contrast, h[Gly2]-GLP-2 had no effect on the levels of caspase-9-like enzymatic activity in irinotecan-treated cells (FIG. 5B). h[Gly2]-GLP-2 also reduced the irinotecan-induced cleavage of the caspase-3 substrate Ac-DEVD-pNA and decreased procaspase-3 cleavage into the active p17 subunit (FIG. 5C). In addition, h[Gly2]-GLP-2 also decreased the irinotecan-induced cleavage of poly-ADP ribose polymerase (PARP), a downstream substrate of activated caspase 3 (FIG. 5D).

### EXAMPLE 6

GLP-2R-dependent Signaling in Irinotecan-induced Apoptosis in vivo and in vitro

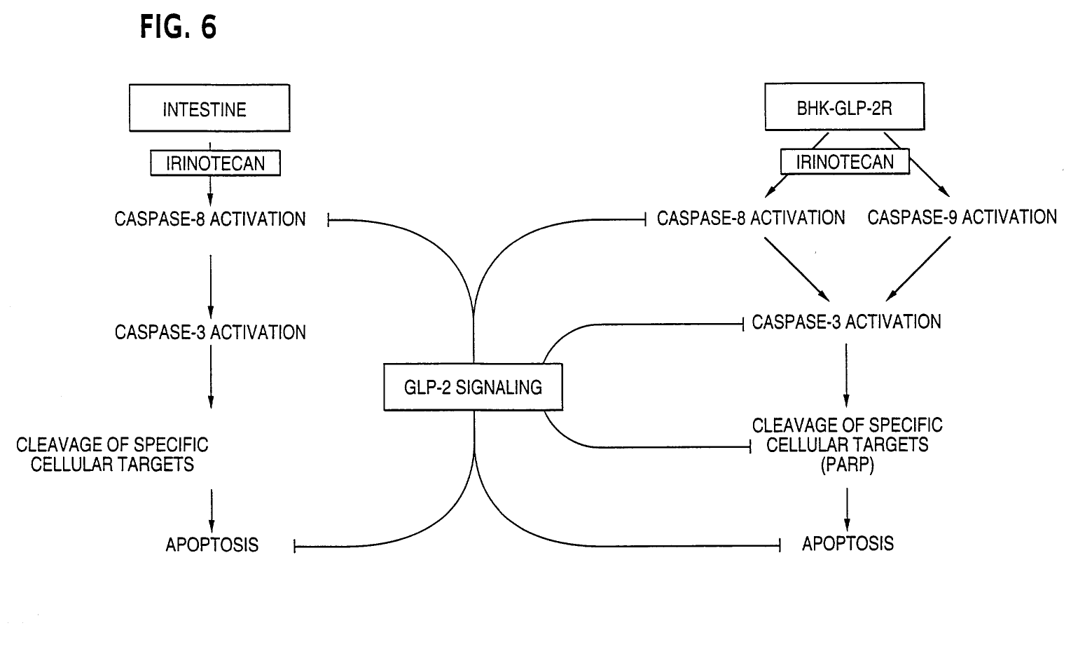

This example is to elucidate the mechanisms activated by GLP-2R signaling to confer resistance to apoptosis-mediated injury in the intestinal epithelium following irinotecan administration. A significant reduction of procaspase 8 cleavage, as evidenced by inhibition of p18 subunit generation, was observed in GLP-2-treated intestine following GLP-2 administration in vivo (FIG. 4C). Reduced irinotecan-mediated activation of caspase-3 enzymatic activity and PARP cleavage in BHK-GLP-2R cells was observed following GLP-2 treatment in vitro (FIG. 5C-D). In contrast, no changes in the levels of caspase 9 enzymatic activity was observed following GLP-2 treatment of BHK-GLP-2R cells in vitro (FIG. 5B). These findings provide new evidence linking direct activation of GLP-2 receptor signaling to specific cell survival pathways in heterologous cell types (FIG. 6).

Although the cellular localization of intestinal GLP-2 receptor expression has not yet been identified, the above data clearly suggests that intestinal cells expressing the GLP-2 receptor are likely to be protected from cell death associated with exposure to genotoxic stress in vitro. Given the emerging importance of GLP-2 receptor signaling for the preservation of intestinal mucosa in the face of intestinal injury (Boushey, R. P. et al., Am. J. Physiol. 277: E937-E947, 1999; Scott, R. B. et al., Am. J. Physiol. 275: G911-G921, 1998; Drucker, D. J. et al., Am. J. Physiol. 276: G79-G91, 1999; Prasad, R. et al., J. Pediatr. Surg. 35: 357-9, 2000), the overall findings from the above examples provide a scientific rationale for exploring the therapeutic use of GLP-2 in settings characterized by induction of intestinal injury via activation in the mucosal epithelium in vivo.

The following materials and methods were specifically employed to obtain the results presented and described in the above examples:

Materials: 5-Fluorouracil (5-FU) was obtained from Roche Laboratories. Irinotecan (7-ethyl-10-[4-(1-piperidino)-1-piperidino] carbonyloxycamptothecin) used in mice was a gift from Pharmacia Upjohn (Mississauga, ON, Canada). Recombinant h[Gly2]-GLP-2 was kindly provided by NPS Allelix Corp. (Mississauga, ON, Canada). Cell culture experiments using irinotecan and forskolin were obtained from Sigma (St. Louis, Mo.).

Animals: All experimental protocols were approved by the Animal Care Committee of the University Health Network-Toronto General Hospital. Experiments with irinotecan alone were performed in 8- to 9-wk old CD1 female mice (Charles River, Canada). Experiments with 5-FU were carried out in 11- to 13-wk old BDF-1 female mice (Harlan, Canada). Experiments using irinotecan treatment in BALB/c mice inoculated with CT-26 murine colon carcinoma cells were performed in 10-wk old female mice (Charles River, Canada). All mice were housed in plastic bottom wire lid cages and maintained in a 12:12-h light-dark cycle temperature-controlled room and given water and chow ad libitum.

Experimental Protocols: All animals were injected with either 0.5 ml saline (PBS) or 10 μg h[Gly2]-GLP-2, a human GLP-2 analog, Drucker, D. J. et al., Nature Biotechnology. 15: 673-677, 1997, dissolved in 0.5 ml saline, twice daily at 08:00 and 18:00 beginning 3 days prior to administration of either 5′-FU (400 mg/kg or irinotecan (280 mg/kg). For non-tumour bearing mice, studies were carried out in adult CD1 female mice. For tumour-bearing BALB/c mice, CT-26 murine colon carcinoma cells (American Tissue Culture Collection) syngeneic to BALB/c mice were grown in monolayer cultures in Dulbecco's Modified Eagle's Medium (DMEM, 4.5 g/l glucose) supplemented with 5% fetal calf serum (FCS), 1 mM pyruvate (Gibco BRL Research Laboratories, Burlington, ON), and penicillin G sodium (100 units/ml)/streptomycin sulfate (0.1 mg/ml) (Sigma, St. Louis, Mo.) in a humidified 5% CO₂ atmosphere at 37° C. as previously described. Shinohara, H., et al. Clin. Cancer Res. 4: 2053-63, 1998. A single cell suspension (exhibiting >90% viability) was injected subcutaneously (5×10⁵ cells) in the left flank region. Six days later, a 7 day treatment regimen consisting of a 3-day treatment with either 0.5 ml saline (PBS) or 10 μg h[Gly2]-GLP-2 was administered twice daily at 08:00 and 18:00 followed by a 3-day regimen of irinotecan (100 mg/kg dose) or vehicle once daily and a 24 hr recovery period. This 7 day regimen was repeated 3 times (n=25 mice per group) at which point some animals in the control groups became moribund hence all mice were euthanized following CO₂ anesthesia 30 days following tumor implantation. The tumour inhibition ratio (IR) was determined using the following equation: IR(%)=(1−T/C)×100 where T and C represent tumour weights in irinotecan-treated (T) and untreated control (C) mice respectively. An IR of 58% was considered to represent an efficacious tumour response to irinotecan. Kunimoto, T. et al., Cancer Res. 47: 5944-7, 1987.

Histological Analysis: Four- to six-micrometer histological cross sections from the intestine of each mouse were cut and stained with hematoxylin and eosin and micrometry was performed as described. Boushey, R. P. et al., Am. J. Physiol. 277: E937-E947, 1999. The number of cells per hemi-crypt column and the number of surviving crypts per circumference were measured in both the small and large intestine at 12 h intervals (n=5 mice per time point) following irinotecan administration as previously described. Wright, N. A. et al., Cell Tissue Kinet. 22: 425-50, 1989; Withers, H. R. et al., Int. J. Radiat. Biol. Relat. Stud. Phys. Chem. Med. 17: 261-7, 1970. Apoptotic cells within the small and large intestinal crypts were scored using the terminal deoxynucleotidyltrans-ferase (TdT) mediated, dUTP-nick end labeling (TUNEL) assay and by their morphological appearance after staining with hematoxylin and eosin. An apoptosis cell index was obtained on a positional basis for all intact half-crypts present in an entire jejunal and colonic cross-section per mouse, as described previously, Pritchard, D. M. et al., Cancer Res. 58: 5453-65, 1998; Potten, C. S. et al., Br. J. Cancer 78: 993-1003, 1998; Ijiri, K. et al., Br. J. Cancer. 47 175-85, 1983, 24 hours following the first dose of irinotecan. Extensive crypt damage precluded an accurate positional analysis of apoptotic events beyond this time point. All slides were scored in a blinded fashion.

Microbiology: At various time points following chemotherapy administration, aliquots of whole blood and tissue homogenates obtained using sterile technique were plated on blood agar plates and incubated at 37° C. for 48 hrs.

Leukocyte Count: Whole blood samples were collected in venipuncture tubes containing EDTA and analyzed using an automated whole blood sorter calibrated for mouse samples. Blood smears were performed on all samples to confirm the automated analysis.

Immunoblotting: Intestinal lysates were centrifuged at 12,000 rpm for 30 min at 4° C. and boiled for 5 minutes in sample buffer. Forty micrograms of total protein were fractionated by discontinuous SDS-PAGE under reducing conditions and electrophoretically transferred onto Hybond-C nitrocellulose membrane (Amersham Pharmacia Biotech, Montreal, Quebec) using standard techniques. Immunoreactive proteins were detected with a secondary antibody conjugated to horseradish peroxidase and an enhanced chemiluminescence commercial kit (Amersham Pharmacia Biotech) as described. Yusta, B. et al., J. Biol. Chem. 274: 30459-67, 1999. Primary antibodies utilized included caspase 3 (1:5000 dilution, gift of R. Sekaly, Montreal), caspases 8 and 9 (both 1:500 dilution, gift of T. Mak, Toronto), p53 (1:500 dilution, PAb 246, Santa Cruz), poly (ADP-ribose) polymerase (PARP) (1:4000 dilution, PharMingen, Canada) and anti-actin (1:5000 dilution, Sigma). Densitometry was performed on blots exposed onto x-ray film (Kodak Diagnostic Film, X-OMAT AR) using a Hewlett Packard ScanJet 3p scanner and the NIH Image software.

Induction of Apoptosis in Transfected BHK Cells: Baby Hamster Kidney (BHK) fibroblast cells containing the stably integrated pcDNA3.1 plasmid (BHK-pcDNA3) (Invitrogen, Carlsbad, Calif.) or the identical plasmid containing the rat GLP-2 receptor (BHK-GLP-2R) were propagated as described. Yusta, B. et al., J. Biol. Chem. 274: 30459-67, 1999. Cells were pretreated with either h[Gly2]-GLP-2 (40 nM) or forskolin (40 μM) prior to the addition of irinotecan (final concentration 10 uM). Control cultures were treated identically in the absence of irinotecan and the number of viable cells in each condition was measured using the Cell-Titer 96 aqueous assay kit (Promega, Madison, Wis.).

Measurement of Caspase-3, -8, and -9 Like Enzymatic Activity: Enzymatic reactions were performed at 37 C. using 150 μg of protein lysate, reaction buffer (Hepes 50 mM (pH 7.4), NaCl 75 mM, CHAPS 0.1%, and DTT 2 mM), and 200 μM of the following substrates: Ac-DEVD-pNA (Calbiochem, San Diego, Calif.) to measure caspase3-like protease activity, Ac-IETD-pNA (Biosource International, Camarillo, Calif.) to measure caspase-8-like protease activity, and Ac-LEHD-pNA (Biosource International) to measure caspase-9-like protease activity, respectively. Spectrophotometric detection of the chromophore paranitroanilide (pNA) at 405 nm was used to quantify enzymatic activity.

We claim:

1. A method for treating a subject having a disease or disorder for which treatment by a chemotherapeutic agent is indicated, the method comprising the steps of:
(i) subjecting the subject to a pretreatment regimen effective to alleviate at least one adverse effect of said chemotherapeutic agent, by administering to the subject a GLP-2 receptor activator each day for a predetermined period of time, and
(ii) within about one week following the pretreatment step (i), subjecting the pretreated subject to a treatment regimen involving chemotherapy administration that is appropriate for treating the disease or disorder.

2. The method according to claim 1, wherein the GLP-2 receptor activator is a GLP-2 receptor agonist.

3. The method according to claim 2, wherein the predetermined period of time is from two days to four days.

4. The method according to claim 3, wherein the predetermined period of time is three consecutive days.

5. The method according to claim 4, wherein the GLP-2 receptor agonist is a GLP-2 analog.

6. The method according to claim 5, wherein the GLP-2 receptor agonist is [GLY]hGLP-2.

7. The method according to claim 2, wherein the GLP-2 receptor agonist is administered to the subject twice a day for three consecutive days.

8. The method according to claim 7, wherein the pretreated subject is subjected to a chemotherapy treatment regimen within about 48 hours following said pretreatment step.

9. The method according to claim 4, wherein the chemotherapeutic agent is selected from 5-Fluorouracil and irinotecan.

10. The method according to claim 9, wherein the GLP-2 receptor agonist is a GLP-2 analog.

11. The method according to claim 10, wherein the GLP-2 receptor agonist is [Gly2]hGLP-2.

12. The method according to claim 4, wherein the pretreatment step is performed using a GLP-2 receptor activator as the sole pretreatment agent.

13. The method according to claim 12, wherein the GLP-2 receptor agonist is a GLP-2 analog.

14. The method according to claim 13, wherein the GLP-2 receptor agonist is [Gly2]hGLP-2.

15. The use of a GLP-2 receptor activator for the pretreatment of a subject having a disease or disorder for which treatment by a chemotherapeutic agent is indicated.

16. The use of a GLP-2 receptor activator according to claim 15, wherein said pretreatment comprises the step of subjecting the subject to a pretreatment regimen effective to alleviate at least one adverse effect of said chemotherapeutic agent, by administering to the subject a GLP-2 receptor activator each day for a pretreatment period sufficient to inhibit chemotherapy-induced activation of apoptosis.

17. The use of a GLP-2 receptor activator acccording to claim 16, wherein said pretreatment period is about 3 days.

18. The use of a GLP-2 receptor activator for the preparation of a medicament useful in a therapeutic regimen to treat a disease or disorder for which treatment by a chemotherapeutic agent is indicated, the therapeutic regimen comprising the steps of:
(i) subjecting the subject to a pretreatment regimen effective to alleviate at least one adverse effect of said chemotherapeutic agent, by administering to the subject a GLP-2 receptor activator each day for a predetermined period of time, and
(ii) within about one week following the pretreatment step (i), subjecting the pretreated subject to a treatment regimen involving chemotherapy administration that is appropriate for treating the disease or disorder.

19. The use of a GLP-2 receptor activator according to claim 18, wherein the GLP-2 receptor activator is administered for a pretreatment period of about three days, and the chemotherapy commences within about one week of said pretreatment.

20. The use of a GLP-2 receptor activator according to claim 19, wherein said GLP-2 receptor activator is a GLP-2 receptor agonist.

21. The use of a GLP-2 receptor activator according to claim 20, wherein said GLP-2 receptor agonist is [Gly2]hGLP-2.
